(19) United States
(12) Patent Application Publication
(10) Pub. No.: US 2017/0260247 A1
(43) Pub. Date: Sep. 14, 2017

(54) Method For Synthesizing Degarelix

(71) Applicant: Chengdu Shengnuo Biopharm Co., Ltd., Chengdu (CN)

(72) Inventors: Dewen Guo, Chengdu (CN); Dezhi Zeng, Chengdu (CN); Guangbin Tong, Chengdu (CN); Yongjun Wen, Chengdu (CN)

(21) Appl. No.: 15/261,085

(22) Filed: Sep. 9, 2016

(30) Foreign Application Priority Data
Mar. 10, 2016 (CN) 201610136374.5

(51) Int. Cl. C07K 14/59; C07K 1/04; C07K 1/12; C07K 1/14; C07K 1/08; C07K 1/107; C07K 1/06

(52) U.S. Cl. CPC C07K 14/592; C07K 1/1077; C07K 1/042; C07K 1/063; C07K 1/14; C07K 1/084; C07K 1/122

(57) ABSTRACT

The present invention relates to the field of medicinal synthesis, and discloses a method for synthesizing degarelix. The method of the present invention as a whole divides the synthesis of degarelix into two parts from amino acids at positions 5 and 6, employs proper protective groups in part of the protected amino acids therein, and finally uses in association with a specific acidolysis agent to complete the whole synthesis process. In the present invention, a proper synthesizing scheme is selected, and adaptive protective group and acidolysis agent are selected, so that the overall synthesis process is optimized, the purity of degarelix is significantly improved with a higer total yield, and the production of the toxic hydantoin degradation product is avoided.

### CROSS REFERENCE TO RELATED APPLICATIONS

The present application claims the priority of Chinese Patent Application No. 201610136374.5, filed on Mar. 10, 2016, the contents of which are incorporated herein by reference in its entirety.

### TECHNICAL FIELD

The present invention relates to the field of medicinal synthesis, and particularly to a method for synthesizing degarelix.

### BACKGROUND

Degarelix is a gonadotropin releasing hormone (GnRH) receptor inhibitor drug developed by Ferring Pharmaceuticals Ltd, Denmark, which reversibly inhibit the GnRH receptor in the hypophysis to reduce the release of gonadotropin, thereby inhibiting the release of testosterone. The present product delays the growth and deterioration of prostate cancer by inhibiting testosterone which is critical for the sustained growth of prostate cancer. During the initial period of treating prostate cancer with hormones to reduce the testosterone concentration, the testosterone concentration is however caused to dramatically increase, which initially stimulates the hormone receptor to temporality promote rather than inhibit the tumor growth, while it is not the case for degarelix. Degarelix was approved for marketing by U.S. FDA on December 2008, which is mainly for patients with advanced prostate cancer, and delays the disease progression of prostate cancer by inhibiting testicular hormones.

It is shown in phase III clinical studies that the effect of degarelix to reduce the testosterone concentration is at least comparable to that of leuprorelin controlled-release injectable depot (Lupron Depot), and the reduction of the testosterone concentration is statistically significantly faster. On day 3 of the treatment, the testosterone concentration in 96% of the group of the present product reaches the testosterone concentration for castration, while in the leuprorelin group, the effect is 0%. On day 14, the testosterone concentration in 99% of the group of the present product reaches the testosterone concentration for castration, while in the leuprorelin group, it was 18%.

In the clinical studies, the concentration of prostate specific antigen (PSA) can act as the second end-point for efficacy judgment in the monitoring. PSA is reduced by 64% following 2 weeks of degarelix administration, 85% following 1 month of degarelix administration, 95% following 3 months of degarelix administration, and PSA is inhibited throughout the whole one year of treatment.

Degarelix has the structural formula of:

Ac-D-Nal-D-Cpa-D-Pal-Ser-Aph(Hor)-D-Aph(Cbm)-Leu-Lys(iPr)-Pro-D-Ala-NH₂.

There are many reports about the report for the preparation of degarelix at home and abroad. U.S. Pat. No. 5,925,730 employs a Boc solid phase synthesis method, wherein the method is on a very small scale, and only the purity of the product is reported to be 98%. Meanwhile, this method needs to use hydrofluoric acid (HF) cleavage, which is more harmful to the human and environment, and thus it is not suitable for large-scale industrial synthesis.

Chinese Patent CN201310336446.7 employs a Fmoc synthesis method, which is also reported to have a very small preparation scale. In order to avoid the production of the toxic degradation products of the rearrangement product hydantoin of Aph(Hor) under alkaline condition, a solid phase segment condensation method as well as unique protected amino acids, Aph(Mmt/Dmt) and Aph(Cbm), are employed in the process. However, it is relatively complicated to manipulate, and the used protected amino acids and segments are expensive, so that the production cost is higher. Chinese Patent CN201210460195.9 employs a Fmoc solid phase synthesis method, which is also reported to have a very small preparation scale. In order to avoid the production of the rearrangement product hydantoin of Aph(Hor) under alkaline condition, a protecting group Trt which can be selectively deprotected, is used for Aph, however TFA/DCM is used in the deprotection process thereof, which will cause Boc in the used protected amino acid Lys(iPr, Boc) to partially lose, thereby reacting the exposed amino in Lys(iPr) with the subsequent L-4,5-dihydroorotic acid to produce new impurities. Meanwhile, it is also relatively complicated to manipulate, and the used protected amino acids are expensive, so that the production cost is higher. In addition, in the above two patents, there is also the problem of low yield of degarelix, which is about 40%. These are related to the overall synthesis process thereof

Chinese Patent CN201410427405.4 employs a Fmoc solid phase synthesis method, which has the simplest manipulation route, and a relatively higher yield than the preceding two Chinese patents. However, the production of degradation products of the rearrangement product hydantoin of Aph(Hor) in the structure under alkaline condition can not be avoided, meanwhile the protected amino acids of the used Aph(Cbm) and Aph(Hor) are also very expensive, so that the cost of the process is higher.

### SUMMARY

In view of this, the objective of the present invention is to provide a new method for synthesizing degarelix, such that the method of the present invention improves the total yield of degarelix and meanwhile avoids the production of the rearrangement product hydantoin, under the premise of ensuring the purity of degarelix.

In order to achieve the above objective, the present invention provides the following technical solutions:

A method for synthesizing degarelix, comprising the following steps:

step 1: esterifying a protected D-alanine with an amino resin having its amino group coupled to a protective group under the action of a condensation reagent and an activation reagent to obtain a peptide resin 1;

step 2: sequentially extending and coupling a protected Pro, protected Lys(ipr), protected Leu and Boc-D-Aph(Fmoc) by starting from the peptide resin 1 according to the order from C-terminus to N-terminus of the amino acid sequence of degarelix under the action of the condensation reagent and the activation reagent, and then removing the side chain Fmoc protective group in D-Aph(Fmoc) to generate D-Aph(NH₂), to obtain a peptide resin 2, wherein the protected Lys(ipr) is a protected Lys(ipr, Z);

step 3: reacting the side chain amino group in D-Aph(NH₂) in the peptide resin 2 with tert-butyl isocyanate under the catalysis of an organic base to generate D-Aph(tBu-Cbm), to obtain a peptide resin 3;

step 4: sequentially extending and coupling a protected Aph(Boc), protected Ser(Bzl), protected D-Pal, protected D-Cpa and protected Ac-D-Nal by starting from the peptide resin 3 according to the order from C-terminus to N-terminus of the amino acid sequence of degarelix under the action of the condensation reagent and the activation reagent, to obtain a peptide resin 4;

step 5: removing the side chain Boc protective group in the protected Aph(Boc) in the peptide resin 4, and incorporating L-4,5-dihydroorotic acid under the action of the condensation reagent and the activation reagent to obtain a degarelix resin;

step 6: acidolysing the degarelix resin by an acidolysis agent to obtain a crude degarelix, wherein the acidolysis agent is a solution of hydrogen bromide in trifluoroacetic acid;

and

step 7: purifying the crude degarelix to obtain a pure degarelix.

Degarelix has 10 main chain amino acids, with a constituent of:

Ac-D-Nal¹-D-Cpa²-D-Pal³-Ser⁴-Aph(Hor)⁵-D-Aph(Cbm)⁶-Leu⁷-Lys(iPr)⁸-Pro⁹-D-Ala¹⁰-NH₂.

Wherein, the amino group at the C-terminus of degarelix is an amino group that is cleaved off from the amino resin by using an acidolysis agent, which does not belong to the amino group on an amino acid.

With respect to the defects in the prior art that the employed synthesis process easily results in a lower yield for degarelix and easily generates the rearrangement product hydantoin, the method of the present invention as a whole divides the synthesis of degarelix into two parts from amino acids at positions 5 and 6, employs proper protective groups in part of the protected amino acids therein, and finally uses in association with a specific acidolysis agent to complete the whole synthesis process. Under the premise to guarantee purity, the total yield of degarelix is improved, and the production of the rearrangement product hydantoin is completely avoided.

The protective groups in the present invention are the protective groups commonly used in the art of synthesis of amino acids that protect synthesis-interfering groups such as an amino, a carboxyl and the like on the main chain and side chain of an amino acid, and prevent the reactions of the amino, carboxyl and the like in the process of preparing the product of interest to generate impurities. For example, in the present invention, the side chain of serine is protected by the protective group Bzl, the side chain of N⁶-(1-methylethyl)lysine (Lys(ipr)) is protected by the protective group Z, and Aph at position 5 is protected by the protective group Boc. In addition, in the protected amino acids involved in the method of the present invention, all the N-terminus is preferably protected by the protective group Fmoc. The amino acid protected by a protective group is referred to as a protected amino acid. Preferably, the protected D-alanine in the step 1 is Fmoc-D-Ala or Boc-D-Ala. The protected Pro, protected Lys(ipr) and protected Leu in the step 2 is:

Fmoc-Pro, Fmoc-Lys(ipr, Z) and Fmoc-Leu; or Boc-Pro, Boc-Lys(ipr, Z) and Boc-Leu.

The protected Aph(Boc), protected Ser(Bzl), protected D-Pal, protected D-Cpa and protected D-Nal is:

Fmoc-Aph(Boc), Fmoc-Ser(Bzl), Fmoc-D-Pal, Fmoc-D-Cpa and Fmoc-D-Nal.

Preferably, the amino resin is MBHA resin.

MBHA resin has the following structural formula, wherein the round ball on the left represents polystyrene resin:

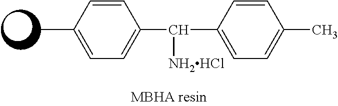

Preferably, the molar ratio of the protected D-alanine to the amino resin having its amino group coupled to a protective group is 1-6:1, more preferably 2.5-3.5:1.

Preferably, the substitution value of the amino resin is 0.2-1.8 mmol/g amino resin, more preferably 0.5-1.0 mmol/g amino resin.

Preferably, the condensation reagent is preferably one of N,N-diisopropyl carbodiimide (DIC), N,N-dicyclohexyl carbodiimide (DCC), benzotriazole-1-yl-oxytripyrrolidinophosphonium hexafluorophosphate/organic base (PyBOP/organic base), 2-(7-aza-1H-benzotriazole-1-yl)-1,1,3,3-tetramethylurea hexafluorophosphate/organic base (HATU/organic base), benzotriazole-N,N,N′,N′-tetramethylurea hexafluorophosphate/organic base (HBTU/organic base), and O-benzotriazole-N,N,N′,N′-tetramethylurea tetrafluoroborate/organic base (TBTU/organic base). The molar amount of the condensation reagent is preferably 1˜6-fold, more preferably 2.5˜3.5-fold of the total moles of amino group in the polypeptide resin.

It is to be specified that the PyBOP/organic base, HATU/organic base, HBTU/organic base and TBTU/organic base in the present invention belong to four types of double-system condensation reagents, i.e., each of PyBOP, HATU and HBTU needs to be combined with an organic base into a condensation reagent when used, wherein the molar ratio of the organic base to the PyBOP, HATU, HBTU and TBTU is preferably 1.3˜3.0:1, more preferably 1.3-2:1.

Preferably, the organic base in the condensation reagent is preferably N,N-diisopropylethylamine (DIPEA), triethylamine (TEA) or N-methylmorpholine (NMM), and more preferably DIPEA.

Preferably, the activation reagent is 1-hydroxybenzotrizole (HOBt) or N-hydroxy-7-azabenzotriazole (HOAt). The amount of the activation reagent is preferably 1˜6-fold, more preferably 2.5˜3.5-fold of the total moles of amino group in the peptide resin.

Preferably, the esterification reaction as well as the reaction for extending and coupling all use DMF as the reaction solvent.

The extending and coupling in the present invention mean that after the coupling of the first amino acid with amino resin, the rest amino acids are condensed (condensation between the main chain amino group and carboxyl group) and coupled with the preceding one coupled amino acid sequentially according to the order from C-terminus to N-terminus of the amino acid sequence of degarelix. During the coupling of the present invention, the molar ratio of the protected amino acid to the corresponding peptide resin during each extending and coupling is preferably 1˜6:1, more preferably 2.5˜3.5:1; the time for coupling is preferably 60˜300 min, more preferably 120˜180 min. It is to be specified that, the peptide resin in the present invention refers to the peptide resin that is formed by the linkage of any number of amino acids and amino resin according to the amino acid order of degarelix, wherein the peptide resin 1 is also included. The corresponding peptide resin refers to the peptide resin 1 formed by coupling D-Ala with amino resin, the peptide resin 5 formed by coupling Pro with the peptide resin 1, the peptide resin 6 formed by coupling Lys(iPr) with the peptide resin 5, the peptide resin 7 formed by coupling Leu with the peptide resin 6, the peptide resin 2 formed by coupling Boc-D-Aph(Fmoc) with the peptide resin 7, the peptide resin 8 formed by coupling Aph(Boc) with the peptide resin 2, the peptide resin 9 formed by coupling Ser with the peptide resin 8, the peptide resin 10 formed by coupling D-Pal with the peptide resin 9, the peptide resin 11 formed by coupling D-Cpa with the peptide resin 10, and the peptide resin 12 formed by coupling D-Nal with the peptide resin 11.

In the extending and coupling, since each amino acid has a protective group at its N-terminus, the protective group at the N-terminus has to be removed before coupling, which is common knowledge for those skilled in the art. In the present invention, a PIP/DMF (piperidine/N,N-dimethylformamide) mixed solution is preferably used to remove the N-terminal Fmoc protective group, wherein the mixed solution contains 10˜30% (V) of piperidine and the balance of DMF. The time for removing the N-terminal protective group is preferably 10˜60 min, preferably 15˜25 min. The amount of the reagent for removing the N-terminal protective group is preferably 10 mL/g peptide resin. In the present invention, a TFA/DCM (trifluoroacetic acid/dichloromethane) mixed solution is preferably used to remove the N-terminal Boc protective group, wherein the mixed solution contains 20˜60% (V/V), preferably 25˜35% (V/V) of trifluoroacetic acid. The time for removing the N-terminal protective group is preferably 10˜50 min, preferably 25˜35 min. The amount of the reagent for removing the N-terminal protective group is preferably 10 mL/g peptide resin. In addition, this preferred scheme is also used to remove the side chain Fmoc protective group in D-Aph(Fmoc) in step 2, and to remove the side chain Boc protective group in Aph(Boc) in peptide resin 4 in step 4.

Preferably, the acidolysis agent is a solution of hydrogen bromide in trifluoroacetic acid (TFA), wherein the concentration in mass percentage of hydrogen bromide is preferably 5˜10% wt, and more preferably 6˜7% wt. The amount of the acidolysis agent is 5˜15 mL acidolysis agent/g peptide resin, preferably 7˜12 mL acidolysis agent/g peptide resin. The time for acidolysis is 1˜6 h, and preferably 3˜4 h.

Preferably, the purification is specifically as following:

The crude degarelix is dissolved with 0.1% TFA/aqueous solution, then the solution is filtered using a 0.45 μm microporous filter membrane and purified, for later use.

High performance liquid chromatography is employed for purification, wherein the chromatographic packing material for purification is reverse phase C18 (10 μm), the mobile phase system is 0.1% TFA/aqueous solution-0.1% TFA/acetonitrile solution, the flow rate of the 77 mm*250 mm chromatographic column is 90 mL/min, a gradient system is employed for elution and the loading is cycled for purification; and wherein the crude solution is loaded onto the chromatographic column, the mobile phase is initiated for elution, the main peak is collected, and the concentrate of the intermediate for degarelix purification is obtained after evaporating acetonitrile.

The concentrate of the intermediate for degarelix purification is taken and filtered using a 0.45 μm filter membrane, for later use.

High performance liquid chromatography is employed for salt exchange, wherein the mobile phase system is 1% acetic acid/aqueous solution-acetonitrile, the chromatographic packing material for purification is reverse phase C18 (10 μm), the flow rate of the 77 mm*250 mm chromatographic column is 90 mL/min, the gradient elution is employed and the loading process is cycled; and wherein the crude solution is loaded onto the chromatographic column, the mobile phase is initiated for elution, the spectrum is collected, the change in absorbance is observed, the main peak for salt exchange is collected and the purity is detected by analyzing the liquid phase, the solutions of the main peak for salt exchange are combined and concentrated under reduced pressure to obtain aqueous acetic acid solution of degarelix, which is freeze-dried to obtain a pure degarelix.

Degarelix synthesized by the method of the present invention is detected by HPLC to have a purity of 99% or more, the largest single impurity is 0.1% or so, no toxic hydantoin degradation products are detected and the total yield is up to 60%. In comparison with synthesis processes for degarelix in the prior art, the process of the present invention can simultaneously reach better levels in terms of purity, total yield and toxic hydantoin degradation products.

As can be seen from the above technical solutions, in the present invention, a proper synthesizing scheme is selected, and adaptive protective group and acidolysis agent are selected, so that the overall synthesis process is optimized, the purity of degarelix is significantly improved with a higher total yield, and the production of toxic hydantoin degradation products is avoided.

### DETAILED DESCRIPTION OF ILLUSTRATIVE EMBODIMENTS

The invention discloses a method for synthesizing degarelix, which can be achieved by those skilled in the art through properly modifying the process parameters in light of the present disclosure. It is specifically to be indicated that, all such similar substitutions and modifications are apparent to those skilled in the art and are deemed to be included in the present invention. The method of the present invention has been described by preferred examples, and it is apparent that the related personnel would achieve and apply the techniques of the present invention by alterations or proper modifications and combinations of the compound and the preparation method described herein, without departing from the content, spirit and scope of the preset invention.

In particular embodiments of the present invention, the amino acids therein are purchased from Chengdu Huirong Biotechnology Co., Ltd., and all the resins are purchased from Shangyu Pure Resin Co., Ltd. The corresponding meanings of the abbreviations used in the application document are listed in Table 1.

TABLE 1
Definitions for abbreviations

| Abbreviation | Name | Abbreviation | Name |
|---|---|---|---|
| Fmoc | 9-fluorenyl- | Aph | 4-amino-phenylalanine |
| | methyloxy- | | |
| | carbonyl | | |
| tBu | tert-butyl | D-Cpa | 4-chloro-D-phenylalanine |
| Boc | tert-butoxy- | D-Ala | D-alanine |
| | carbonyl | | |
| Bzl | benzyl | D-Aph | 4-amino-D-phenylalanine |
| Z | carbobenzoxy | Lys(iPr) | N⁶-(1-methylethyl)lysine |
| Ser | serine | D-Nal | 3-(2-naphthyl)-D-alanine |
| Leu | leucine | D-Pal | 3-(3-pyridyl)-D-alanine |
| Ac | acetyl | DIEA | |

Hereinafter, the present invention will be further explained in conjunction with Examples.

### Example 1: Synthesis of the Peptide Resin 1

0.15 mol Fmoc-D-Ala and 0.15 mol HOBt were taken, and dissolved with an appropriate amount of DMF. 0.15 mol DIC was additionally taken, and slowly added to the solution of the protected amino acid in DMF under stirring. The reaction was stirred for 30 min at room temperature to obtain the activated protected amino acid solution for later use.

0.05 mol MBHA resin (with a substitution value of about 0.6 mmol/g) was taken, swollen in DMF for 25 min, washed and filtered. The activated Fmoc-D-Ala solution was added and the reaction was stirred for 3 h at room temperature. The reaction solution was sucked away, washed with DMF for three times and then with DCM for 3 times (3 min for each wash) to obtain Fmoc-D-Ala-MBHA resin, which was deprotected with 20% PIP/DMF solution for 25 min, washed and filtered to obtain the peptide resin 1 (D-Ala-MBHA resin).

### Example 2: Synthesis of the Peptide Resin 1

0.15 mol Boc-D-Ala and 0.15 mol HOBt were taken, and dissolved with an appropriate amount of DMF. 0.15 mol DIC was additionally taken, and slowly added to the solution of the protected amino acid in DMF under stirring. The reaction was stirred for 30 mm at room temperature to obtain the activated protected amino acid solution for later use.

0.05 mol MBHA resin (with a substitution value of about 0.6 mmol/g) was taken, swollen in DMF for 25 min, washed and filtered. The activated Fmoc-D-Ala solution was added and the reaction was stirred for 3 h at room temperature. The reaction solution was sucked away, washed with DMF for 3 times and then with DCM for 3 times (3 min for each wash) to obtain Boc-D-Ala-MBHA resin, which was deprotected with 30% TFA/DCM solution for 30 min, neutralized with DIEA/DCM solution, washed with DMF, DCM and filtered to obtain the peptide resin 1 (D-Ala-MBHA resin).

### Example 3: Synthesis of the Peptide Resin 2

0.15 mol Fmoc-Pro and 0.15 mol HOBt were taken, and dissolved with an appropriate amount of DMF. 0.15 mol DIC was additionally taken, and slowly added to the solution of the protected amino acid in DMF under stirring. The reaction was stirred for 30 mm at room temperature to obtain the activated protected amino acid solution.

The activated protected amino acid solution described above was added to the peptide resin 1 prepared in Example 1, and the reaction was stirred for 3 h at room temperature. The reaction solution was sucked away, washed with DMF for 3 times and then with DCM for 3 times (3 min for each wash), deprotected again with 20% PIP/DMF solution for 25 min, washed and filtered to complete the incorporation of Pro.

Fmoc-Lys(ipr, Z), Fmoc-Leu and Boc-D-Aph(Fmoc) were incorporated by the same process, and then finally deprotected again with 20% PIP/DMF solution to form the peptide resin 2

[Boc-D-Aph(NH₂)-Leu-Lys(ipr, Z)-Pro-D-Ala-MBHA resin].

### Example 4: Synthesis of the Peptide Resin 2

0.15 mol Boc-Pro and 0.15 mol HOBt were taken, and dissolved with an appropriate amount of DMF. 0.15 mol DIC was additionally taken, and slowly added to the solution of the protected amino acid in DMF under stirring. The reaction was stirred for 30 min at room temperature to obtain the activated protected amino acid solution.

The activated protected amino acid solution described above was added to the peptide resin 1 prepared in Example 2, and the reaction was stirred for 3 h at room temperature. The reaction solution was sucked away, washed with DMF for 3 times and then with DCM for 3 times (3 min for each wash), deprotected again with 30% TFA/DCM solution for 30 min, neutralized with DIEA/DCM solution, washed with DMF, DCM and filtered to complete the incorporation of Pro.

The incorporation of Boc-Lys(ipr, Z) and Boc-Leu was completed by the same process.

0.15 mol Boc-D-Aph(Fmoc) and 0.15 mol HOBt were taken, and dissolved with an appropriate amount of DMF. 0.15 mol DIC was additionally taken, and slowly added to the solution of the protected amino acid in DMF under stirring. The reaction was stirred for 30 min at room temperature to obtain the activated protected amino acid solution, which was added to the above resin which had completed the incorporation of Pro, Boc-Lys(ipr, Z) and Boc-Leu, and the reaction was stirred for 3 h at room temperature. The reaction solution was sucked away, washed with DMF for 3 times and then with DCM for 3 times (3 min for each wash), and deprotected again with 20% PIP/DMF solution to obtain the peptide resin 2 [Boc-D-Aph(NH₂)-Leu-Lys(ipr, Z)-Pro-D-Ala-MBHA resin].

### Example 5: Synthesis of the Peptide Resin 3

0.5 mol tert-butyl isocyanate and 0.5 mol DIEA were taken, dissolved with an appropriate amount of DMF, and added to the peptide resin 2 prepared in Example 3. The reaction was stirred at room temperature overnight. The reaction solution was sucked away, washed with DMF for 3 times and then with DCM for 3 times (3 min for each wash), and deprotected again with 30% TFA/DCM solution for 30 min, washed with DMF, DCM and filtered to obtain the peptide resin 3 [NH₂-D-Aph(tBu-Cbm)-Leu-Lys(ipr, Z)-Pro-D-Ala-MBHA resin].

### Example 6: Synthesis of the Peptide Resin 3

0.5 mol tert-butyl isocyanate and 0.5 mol DIEA were taken, dissolved with an appropriate amount of DMF, and added to the peptide resin 2 prepared in Example 4. The reaction was stirred at room temperature overnight. The reaction solution was sucked away, washed with DMF for 3 times and then with DCM for 3 times (3 min for each wash), and deprotected again with 30% TFA/DCM solution for 30 min, neutralized with DIEA/DCM solution, washed with DMF, DCM and filtered to obtain the peptide resin 3 [NH₂-D-Aph(tBu-Cbm)-Leu-Lys(ipr, Z)-Pro-D-Ala-MBHA resin].

### Example 7: Synthesis of the Peptide Resin 4

0.15 mol Fmoc-Aph(Boc) and 0.15 mol HOBt were taken, and dissolved with an appropriate amount of DMF. 0.15 mol DIC was additionally taken, and slowly added to the solution of the protected amino acid in DMF under stirring. The reaction was stirred for 30 min at room temperature to obtain the activated protected amino acid solution.

The activated protected amino acid solution above was added to the peptide resin 3 prepared in Example 5. The reaction was stirred for 3 h at room temperature. The reaction solution was sucked away, washed with DMF for 3 times and then with DCM for 3 times (3 min for each wash), deprotected again with 20% PIP/DMF solution for 25 min, washed and filtered to complete the incorporation of Fmoc-Aph(Boc).

Fmoc-Ser(Bzl), Fmoc-D-Pal, Fmoc-D-Cpa, Fmoc-D-Nal and Ac₂O were incorporated by the same process, then deprotected again with 30% TFA/DCM solution for 30 min, neutralized with DIEA/DCM solution, washed with DMF, DCM and filtered to obtain the peptide resin 4 [Ac-D-Nal-D-Cpa-D-Pal-Ser(Bzl)-Aph(Boc)-D-Aph(tBu-Cbm)-Leu-Lys(ipr, Z)-Pro-D-Ala-MBHA resin].

### Example 8: Synthesis of the Peptide Resin 4

0.15 mol Fmoc-Aph(Boc) and 0.15 mol HOBt were taken, and dissolved with an appropriate amount of DMF. 0.15 mol DIC was additionally taken, and slowly added to the solution of the protected amino acid in DMF under stirring. The reaction was stirred for 30 min at room temperature to obtain the activated protected amino acid solution.

The activated protected amino acid solution described above was added to the peptide resin 3 prepared in Example 6. The reaction was stirred for 3 h at room temperature. The reaction solution was sucked away, washed with DMF for 3 times and then with DCM for 3 times (3 min for each wash), deprotected again with 20% PIP/DMF solution for 25 min, washed and filtered to complete the incorporation of Fmoc-Aph(Boc).

Fmoc-Ser(Bzl), Fmoc-D-Pal, Fmoc-D-Cpa, Fmoc-D-Nal and Ac₂O were incorporated by the same process, then deprotected again with 30% TFA/DCM solution for 30 min, neutralized with DIEA/DCM solution, washed with DMF, DCM and filtered to obtain the peptide resin 4 [Ac-D-Nal-D-Cpa-D-Pal-Ser(Bzl)-Aph(Boc)-D-Aph(tBu-Cbm)-Leu-Lys(ipr, Z)-Pro-D-Ala-MBHA resin].

### Example 9: Synthesis of the Degarelix Peptide Resin

0.2 mol L-4,5-dihydrooratic acid (Hor) and 0.2 mol HOBt were taken, and dissolved with an appropriate amount of DMF. 0.2 mol DIC was additionally taken, and slowly added to the solution of the protected amino acid in DMF under stirring. The reaction was stirred for 30 min at room temperature to obtain the activated protected amino acid solution.

The activated protected amino acid solution described above was added to the peptide resin 4 prepared in Example 7. The reaction was stirred for 6 h at room temperature. The reaction solution was sucked away, washed with DMF for 3 times and then with DCM for 3 times (3 min for each wash) to obtain the degarelix peptide resin [Ac-D-Nal-D-Cpa-D-Pal-Ser(Bzl)-Aph(Hor)-D-Aph(tBu-Cbm)-Leu-Lys(ipr, Z)-Pro-D-Ala-MBHA resin].

### Example 10: Synthesis of the Degarelix Peptide Resin

0.2 mol L-4,5-dihydrooratic acid (Hor) and 0.2 mol HOBt were taken, and dissolved with an appropriate amount of DMF. 0.2 mol DIC was additionally taken, and slowly added to the solution of the protected amino acid in DMF under stirring. The reaction was stirred for 30 min at room temperature to obtain the activated protected amino acid solution.

The activated protected amino acid solution described above was added to the peptide resin 4 prepared in Example 8. The reaction was stirred for 6 h at room temperature. The reaction solution was sucked away, washed with DMF for 3 times and then with DCM for 3 times (3 min for each wash) to obtain the degarelix peptide resin [Ac-D-Nal-D-Cpa-D-Pal-Ser(Bzl)-Aph(Hor)-D-Aph(tBu-Cbm)-Leu-Lys(ipr, Z)-Pro-D-Ala-MBHA resin].

### Example 11: Preparation of the Crude Degarelix

The degarelix peptide resin prepared in Example 9 was taken, and 8% of HBr/TFA solution (acidolysis solution 10 mL/g degarelix peptide resin) was added. The reaction was stirred for 6 h, and filtered to collect the filtrate. The resin was again washed with a small amount of TFA for 3 times. The filtrates were combined and concentrated under reduced pressure. Anhydrous diethyl ether was added to precipitate, and the precipitate was washed with anhydrous diethyl ether again for 3 times, and sucked dry to obtain an off-white power, i.e. the crude degarelix, which had a purity of 86.7%.

### Example 12: Preparation of the Crude Degarelix

The degarelix peptide resin prepared in Example 10 was taken, and 8% of HBr/TFA solution (acidolysis solution 10 mL/g degarelix peptide resin) was added. The reaction was stirred for 6 h, and filtered to collect the filtrate. The resin was again washed with a small amount of TFA for 3 times. The filtrates were combined and concentrated under reduced pressure. Anhydrous diethyl ether was added to precipitate, and the precipitate was washed with anhydrous diethyl ether again for 3 times, and sucked dry to obtain an off-white power, i.e. the crude degarelix, which had a purity of 84.0%.

### Example 13: Purification of the Crude Degarelix

The crude degarelix obtained in Example 11 was taken and dissolved with 20% acetic acid solution. The solution was filtered using a 0.45 μm microporous filter membrane and used for purification.

High performance liquid chromatography was employed for purification. The chromatographic packing material for purification was reverse phase C18 (10 μm), the mobile phase system was 0.1% TFA/aqueous solution-0.1% TFA/acetonitrile solution, the flow rate of the 77 mm*250 mm chromatographic column was 90 mL/min, a gradient system was employed for elution and the loading was cycled for purification. The crude solution was loaded onto the chromatographic column, the mobile phase was initiated for elution, the main peak was collected, and the concentrate of the intermediate for degarelix purification was obtained after evaporating acetonitrile.

The concentrate of the intermediate for degarelix purification was taken and filtered using a 0.45 μm filter membrane, for later use.

High performance liquid chromatography was employed for salt exchange. The mobile phase system was 1% acetic acid/aqueous solution-acetonitrile, the chromatographic packing material for purification was reverse phase C18 (10 μm), the flow rate of the 77 mm*250 mm chromatographic column was 90 mL/min, the gradient elution was employed and the loading process was cycled. The crude solution was loaded onto the chromatographic column, the mobile phase was initiated for elution, the spectrum was collected, the change in absorbance was observed, the main peak for salt exchange was collected and the purity was detected by analyzing the liquid phase, the solutions of the main peak for salt exchange were combined and concentrated under reduced pressure to obtain aqueous degarelix acetate, which was freeze-dried to obtain the pure degarelix, 49.3 g.

The total yield was 60.4%, the molecular weight was 1633.0, the purity was 99.5%, the largest single impurity was 0.10%, and no toxic hydantoin degradation product was detected.

### Example 14: Purification of the Crude Degarelix

The crude degarelix obtained in Example 12 was taken and dissolved with mobile phase A for purification. The solution was filtered using a 0.45 μm microporous filter membrane and purified, for later use.

High performance liquid chromatography was employed for purification. The chromatographic packing material for purification was reverse phase C18 (10 μm), the mobile phase system was 0.1% TFA/aqueous solution-0.1% TFA/acetonitrile solution, the flow rate of the 77 mm*250 mm chromatographic column was 90 mL/min, a gradient system was employed for elution and the loading was cycled for purification. The crude solution was loaded onto the chromatographic column, the mobile phase was initiated for elution, the main peak was collected, and the concentrate of the intermediate for degarelix purification was obtained after evaporating acetonitrile.

The concentrate of the intermediate for degarelix purification was taken and filtered using a 0.45 μm filter membrane, for later use.

High performance liquid chromatography was employed for salt exchange. The mobile phase system was 1% acetic acid/aqueous solution-acetonitrile, the chromatographic packing material for purification was reverse phase C18 (10 μm), the flow rate of the 77 mm*250 mm chromatographic column was 90 mL/min, the gradient elution was employed and the loading process was cycled. The crude solution was loaded onto the chromatographic column, the mobile phase was initiated for elution, the spectrum was collected, the change in absorbance was observed, the main peak for salt exchange was collected and the purity was detected by analyzing the liquid phase, the solutions of the main peak for salt exchange were combined and concentrated under reduced pressure to obtain aqueous acetic acid solution of degarelix, which was freeze-dried to obtain the pure degarelix, 47.5 g.

The total yield was 58.2%, the molecular weight was 1633.4, the purity was 99.7%, the largest single impurity was 0.11%, and no toxic hydantoin degradation product was detected.

The above described is merely preferred embodiments of the present invention. It should be noted that, an ordinary skilled in the art can make various improvements and modifications without departing from the principle of the present invention, and these improvements and modifications are also deemed to be within the protection scope of the present invention.

1. A method for synthesizing degarelix, comprising the following steps:
step 1: condensing a protected D-alanine with an amino resin under the action of a condensation reagent and an activation reagent to obtain a peptide resin 1;
step 2: sequentially extending and coupling a protected Pro, protected Lys(ipr), protected Leu and Boc-D-Aph(Fmoc) by starting from the peptide resin 1 according to the order from C-terminus to N-terminus of the amino acid sequence of degarelix under the action of the condensation reagent and the activation reagent, and then removing the side chain Fmoc protective group in Aph(Fmoc) to generate D-Aph(NH₂), to obtain a peptide resin 2, wherein the protected Lys(ipr) is a protected Lys(ipr, Z);
step 3: reacting the side chain amino group in D-Aph(NH₂) in the peptide resin 2 with tert-butyl isocyanate under the catalysis of an organic base to generate D-Aph(tBu-Cbm), to obtain a peptide resin 3;
step 4: sequentially extending and coupling a protected Aph(Boc), protected Ser(Bzl), protected D-Pal, protected D-Cpa, protected D-Nal and Ac₂O by starting from the peptide resin 3 according to the order from C-terminus to N-terminus of the amino acid sequence of degarelix under the action of the condensation reagent and the activation reagent, to obtain a peptide resin 4;
step 5: removing the side chain Boc protective group in the protected Aph(Boc) in the peptide resin 4, and incorporating L-4,5-dihydroorotic acid under the action of the condensation reagent and the activation reagent to obtain a degarelix resin;
step 6: acidolysing the degarelix resin by an acidolysis agent to obtain a crude degarelix, wherein the acidolysis agent is a solution of hydrogen bromide in trifluoroacetic acid; and
step 7: purifying the crude degarelix to obtain a pure degarelix.

2. The method according to claim 1, wherein the protected D-alanine in step 1 is Fmoc-D-Ala or Boc-D-Ala.

3. The method according to claim 1, wherein the protected Pro, protected Lys(ipr) and protected Leu in step 2 is Fmoc-Pro, Fmoc-Lys(ipr, Z) and Fmoc-Leu; or Boc-Pro, Boc-Lys(ipr, Z) and Boc-Leu.

4. The method according to claim 1, wherein the amino resin is MBHA resin.

5. The method according to claim 1, wherein the molar ratio of the protected D-alanine to the amino resin having its amino group coupled to a protective group is 1-6:1.

6. The method according to claim 1, wherein the condensation reagent is one of N,N-diisopropyl carbodiimide, N,N-dicyclohexyl carbodiimide, benzotriazole-1-yl-oxytripyrrolidinophosphonium hexafluorophosphate/organic base, 2-(7-aza-1H-benzotriazole-1-yl)-1,1,3,3-tetramethylurea hexafluorophosphate/organic base, benzotriazole-N,N,N′,N′-tetramethylurea hexafluorophosphate/organic base, and O-benzotriazole-N,N,N′,N′-tetramethylurea tetrafluoroborate/organic base.

7. The method according to claim 1, wherein the organic base is N,N-diisopropylethylamine, triethylamine or N-methylmorpholine.

8. The method according to claim 1, wherein the activation reagent is 1-hydroxybenzotrizole or N-hydroxy-7-azabenzotriazole.

9. The method according to claim 1, wherein the concentration in mass percentage of hydrogen bromide in the acidolysis agent is 5%40%.

10. The method according to claim 1, wherein the protected Aph(Boc), protected Ser(Bzl), protected D-Pal, protected D-Cpa and protected D-Nal are Fmoc-Aph(Boc), Fmoc-Ser(Bzl), Fmoc-D-Pal, Fmoc-D-Cpa and Fmoc-D-Nal.
